(19) United States
(12) Patent Application Publication
(10) Pub. No.: US 2016/0123975 A1
(43) Pub. Date: May 5, 2016

(54) Mesh Microfluidic Mixing Chamber

(71) Applicant: Daktari Diagnostics, Inc., Cambridge, MA (US)

(72) Inventors: Robert Etheredge, Natick, MA (US); Yevgeny Yurkovetsky, Winchester, MA (US); Aaron Oppenheimer, Cambridge, MA (US)

(21) Appl. No.: 14/929,705

(22) Filed: Nov. 2, 2015

Related U.S. Application Data
(60) Provisional application No. 62/074,347, filed on Nov. 3, 2014.

(51) Int. Cl. G01N 33/543; G01N 33/569

(52) U.S. Cl. CPC G01N 33/54386; G01N 33/56983

(57) ABSTRACT

This document provides devices, systems, and methods for detecting pathogens in a biological sample. In some cases, the devices, systems, and methods include a microfluidic device, which includes a biological sample inlet in fluid communication with at least a first microfluidic chamber. The first microfluidic chamber holds a fibrous matrix. The fibrous matrix can carrying anticoagulant and target capture agents within the fibrous matrix. In some cases, the fibrous matrix can be a nonwoven sheet, which can entangle and retain particles bonded to the target capture agents. When in use, a biological sample is introduced into a microfluidic device to flow through the fibrous matrix to mix with the anticoagulant and contact target capture agents. The target capture agents can either remain in the fibrous matrix or flow to a target capture agent chamber. A lysis agent can be delivered through microfluidic chambers including the target capture agent and any captured targets to lyse the target and produce lysate, which can be analyzed to detect a presence of the target in the biological sample.

### CROSS-REFERENCE TO RELATED APPLICATIONS

This application claims the benefit of U.S. Provisional Application Ser. No. 62/074,347, filed Nov. 3, 2014. The disclosure of the prior application is considered part of (and is incorporated by reference in) the disclosure of this application.

### TECHNICAL FIELD

This document relates to devices, systems, and methods involved in the detection of pathogens in bodily fluids such as blood. For example, this document provides microfluidic designs including microchambers including mesh structures including anticoagulant and target capture agents.

### BACKGROUND

In parts of the world, diseases such as Hepatitis C, HIV infection (and various stages of the disease), syphilis infection, malaria infection, and anemia are common and debilitating to humans, particularly to pregnant women. For example, Hepatitis C is an infectious disease affecting primarily the liver, caused by the hepatitis C virus (HCV). An HCV infection can be asymptomatic. A chronic HCV infection, however, can lead to scarring of the liver and ultimately to cirrhosis, which can result in liver failure, liver cancer, or even deadly esophageal and gastric varices. Accordingly, there is a need to detect the presence of HCV prior to an infected person becoming symptomatic. Likewise, there is also a need to detect other pathogens, such HIV, syphilis, and malaria, prior to an infected person becoming symptomatic. Currently available detection procedures, however, are costly and/or time consuming. Moreover, point-of-care medical diagnostic tools can require complex arrangements for the mixing of samples and reagents and dispersed phase transfer in order to achieve a sufficient sensitivity for the detection of pathogens in collected biological samples. These complex arrangements add to the cost of design, development, and manufacturing these tools.

### SUMMARY

This document provides devices, systems, and methods for reducing the complexities of reagent mixing in a microfluidic architecture. Devices, systems and methods provided herein can improve the mixing of liquids, liquids and solids, and provide scaffolds for dispersed phase t that also allow transfer in a microfluidic architecture. Devices, systems and methods provided herein can reduce and/or improve the accuracy/sensitivity of a pathogen detection apparatus.

In some aspects, a pathogen detection apparatus provided herein includes a fibrous matrix carrying an anticoagulant and target capture agents. In some cases, the devices, systems, and methods include a microfluidic device, which can include a biological sample inlet in fluid communication with at least a first microfluidic chamber. The at least a first microfluidic chamber can hold a fibrous matrix. In some cases, the fibrous matrix can be a nonwoven sheet, which can entangle and retain particles bonded to the target capture agents. In some cases, the target capture agents can be virion capture agents. In some cases, the target capture agents can be bound to particles retained in the fibrous matrix (e.g., retained in a non-woven mesh). In some cases, the particles can be nanoparticles. In some cases, the particles can be metallic. In some cases, the particles can be magnetic. In some cases, the fibrous matrix (e.g., a non-woven mesh) can be positioned inside a sample intake chamber of the pathogen detection apparatus. In some cases, the fibrous matrix can include fibers that are entangled, thermally bonded, chemically bonded, layered, or a combination thereof. In some cases, the fibrous matrix can be a sheet of non-woven material.

In some aspects, a pathogen detection apparatus provided herein includes a fibrous matrix retaining a plurality of target capture agents. In some cases, the fibrous matrix can be a non-woven mesh. In some cases, the devices, systems, and methods include a microfluidic device, which can include a biological sample inlet in fluid communication with at least a first microfluidic chamber. The at least a first microfluidic chamber can hold a fibrous matrix. In some cases, the fibrous matrix can be a nonwoven sheet, which can entangle and retain particles bonded to the target capture agents. In some cases, the target capture agents can be virion capture agents. In some cases, the target capture agents can be bound to particles retained in the fibrous matrix (e.g., retained in a non-woven mesh). In some cases, the particles can be nanoparticles. In some cases, the particles can be metallic. In some cases, the particles can be magnetic. In some cases, the fibrous matrix (e.g., a non-woven mesh) can be positioned inside a sample intake chamber of the pathogen detection apparatus. In some cases, the fibrous matrix can include fibers that are entangled, thermally bonded, chemically bonded, layered, or a combination thereof. In some cases, the fibrous matrix can be a sheet of non-woven material.

The fibrous matrix can include fibers made out of any suitable material and having any suitable fiber dimensions. Suitable materials include materials that are biologically inert. In some cases, the fibers can be hydrophobic. In some cases, the fibers can be hydrophilic. In some cases, the fibers can include both hydrophilic and hydrophobic materials. In some cases, fibers can be engineered, coated, or finished to allow binding or capture of certain targets. Blood constituents like platelets might bind to the fibers and reduce the effectiveness of the fibrous matrix so the fibers need to be modified chemically. An example of this is the use of phosphorylcholine-like coatings that present a surface that platelets identify as endothelium and pass through without binding. In some cases, the fibrous matrix can include polypropylene (PP), polyester (PET) and copolyesters, polyamide, and copolyamides, cellulosics, rayon, and bi/multicomponents fibers. In some cases, the fibers can have a fiber length of 1-75 mm for natural fibers and continuous length for synthetic fibers such as PP and PET that are melt spun or melt blown into continuous webs of 5 meters wide. In some cases, the fibers can have a fiber diameter of between 0.1 microns and 15 microns with commercial diameters of around 10-15 microns usually found. The fact that polymeric fibers are extruded through rows of spinnerets, elongated during manufacture to promote variable tensile properties, and deposited into various custom entanglement webs allows enormous design capability.

The fibrous matrix can be a sheet of non-woven material made using any suitable process. In some cases, the fibrous matrix can be produced by air laying or wet laying natural fibers, spinning synthetic fibers, and bonding and/or entangling layers of fibers. In some cases, the fibrous matrix can be produced by melt blowing polymeric fibers. In some cases, the fibrous matrix can be produced by e-spinning material into fibers and randomly laying down the fibers. Preferred materials for this application are PP and PET using processes of spinning, thermal bonding, needle punching, and spun lacing, The sheet of non-woven material can have a basis weight of 5-800 grams/sq. meter with preferred basis weight of 10-100 grams/sq. meter, In some cases, the basis weight, void volume, and average pore size is selected to retain a majority of target capture agents within the non-woven web. In some cases, target capture agents are bound to particles (e.g., nanoparticles) retained within the non-woven web and the basis weight, void volume, and average pore size of the non-woven web are selected to retain at least 80% of the particles when one liter of water flows through the non-woven web. In some cases, the particles retained within the non-woven web can have an average particle size greater than the average pore size of the non-woven web. In some cases, the fibrous matrix can be purchased from a nonwoven supplier, such as DelStar and Freudenberg.

In some cases, a pathogen detection device provided herein includes particles retained within a fibrous matrix. In some cases, the particles can be immobilized in the fibrous matrix. In some cases, the particles are immobilized physically. For example, particles can be captured through filtration using the nonwoven matrix. In some cases, particles can be intermixed within the polymeric network and dried in place. In some cases, the particles are immobilized chemically. For example, in some cases, particles can bonded to polymeric fibers. In some cases the binding moiety can be chemically applied to the fiber surfaces of the fibrous web thereby increasing the active surface area for capture by hundreds of times. In some cases, the particles are immobilized magnetically. For example, particles can be attracted to a magnet positioned relative to the fibrous matrix to limit migration of the particles within the fibrous matrix. The particles can have any suitable size or shape. The particles can include any suitable material. In some cases, a target capture agent (e.g., a virion capture agent) is bound (e.g., covalently bound) to the particles. In some cases, the particles are metallic. In some cases, the particles are magnetic. In some cases, the particles can include silver. For example, core antigen can be captured in the presence of silver nanoparticles that can further be dissociated into silver ions used to quantify the core antigen.

Pathogen detection devices provided herein can be used to isolate and detect target molecules or parts of molecules. In some cases, a biological sample and an anticoagulant are introduced to a microfluidic chamber including a fibrous matrix provided herein. In some cases, the biological sample and the buffer can be introduced through the same port. In some cases, the biological sample and the buffer can be introduced through different ports. In some cases, the biological sample and the buffer are introduced to the microfluidic chamber at the same time. In some cases, the biological sample and the buffer can intermix while in the microfluidic chamber. In some cases, targets (e.g., cells, viruses, proteins, antibodies, macromolecules, DNA, RNA) can bind with target capture agents retained in the fibrous matrix. In some cases, a mixture of biological sample and the buffer can exit the microfluidic chamber through an exit port. In some cases the biological sample can mix with the anticoagulant and target capture agent in the microfluidic chamber followed by a rinse step and then followed by lysis reagent within the same chamber. Once lysis is complete, the core antigen of the bound virions can be transported out of the compartment for further processing and quantitation. In some cases, a rinse buffer can be introduced after the biological sample but prior to the introduction of a lysis buffer.

In some aspects, a method of detecting the presence of a target in a biological sample includes introducing a biological sample into a microfluidic device to flow through the fibrous matrix to mix with the anticoagulant and contact target capture agents. The target capture agents can either remain in the fibrous matrix or flow to a target capture agent chamber. A lysis agent can be delivered through microfluidic chambers including the target capture agent and any captured targets to lyse the target and produce lysate, which can be analyzed to detect a presence of the target in the biological sample.

A method for running a diagnostic analysis provided herein can include delivering a blood sample to a cartridge including a fibrous matrix provided herein, inserting the cartridge into a controller, and activating the controller to run a diagnostic analysis, where the diagnostic analysis includes a step of delivering a rinse buffer and/or a lysis buffer from one or more reservoirs on the cartridge.

The details of one or more embodiments are set forth in the accompanying drawings and the description below. Other features and advantages will be apparent from the description and drawings, and from the claims.

### DESCRIPTION OF DRAWINGS

FIG. 1 depicts an example of a first microfluidic arrangement including a chamber including a fibrous matrix provided herein.

FIG. 2 depicts an example of a second microfluidic arrangement including multiple microfluidic chambers provided herein.

FIG. 3 is a flow chart describing how microfluidic arrangements provided herein can be used to produce lysate.

FIG. 4 depicts an example of a third microfluidic arrangement including multiple microfluidic chambers a target capture agent collection chamber including a magnet.

FIG. 5 depicts an example of a third microfluidic arrangement including multiple microfluidic chambers and target capture agents flow to a target capture agent collection chamber including a capture fibrous matrix.

### DETAILED DESCRIPTION

This document provides systems, methods, and devices related to mixing biological samples with anticoagulant and collecting targets with target capture agents. In some cases, anticoagulant and/or target capture agents are retained in a fibrous matrix. In some cases, microfluidic devices provided herein can be used to receive a biological sample (e.g., a finger prick of blood) and sufficiently mix the sample with anticoagulant. By retaining anticoagulant in the fibrous matrix in a microfluidic chamber, the biological sample can wick through the fibrous matrix and simultaneously intermix with dispersed anticoagulant in the fibrous matrix. The mixing of fluids in a microfluidic channel can require a complex and tortuous arrangement to force the fluids to intermix, thus the use of a fibrous matrix holding an anticoagulant can simplify a design for a biological sample receiving microfluidic device where the sample needs to be intermixed with a reagent, buffer, or other additive. Accordingly, the use of a fibrous matrix holding at least anticoagulant in a microfluidic device used to analyze a biological sample and/or detect a target constituent can provide more efficient and more accurate data.

In some cases, a fibrous matrix can include target capture agents. In some cases, a single fibrous matrix can include both anticoagulant and target capture agents. In some cases, an initial fibrous matrix can include anticoagulant and a subsequent fibrous matrix can include target capture agent. In some cases, methods, devices, and systems provided herein can be adapted for use with sample either premixed with anticoagulant or that do not require anticoagulant, thus certain embodiments of microfluidic devices provided herein might not include anticoagulant in a fibrous matrix.

In some cases, target capture agents can be held and retained in at least one fibrous matrix such that at least 50 weight percent of the target capture agents remain in the fibrous matrix after the sample, a rinsing buffer, and a lysis buffer passes through the fibrous matrix. In some cases, at least 60 weight percent, at least 70 weight percent, at least 80 weight percent, at least 90 weight percent, or at least 95 weight percent of the target capture agents remain in the fibrous matrix after the sample, a rinsing buffer, and a lysis buffer passes through the fibrous matrix. Target capture agents can be retained in a fibrous matrix by bonding target capture agents to particles (e.g., nanoparticles). The nanoparticles can be retained within the fibrous matrix physically, chemically, or magnetically. Some examples of how the particles can be retained are discussed below. In some cases, target capture agents can be bonded directly to fibers of the fibrous matrix.

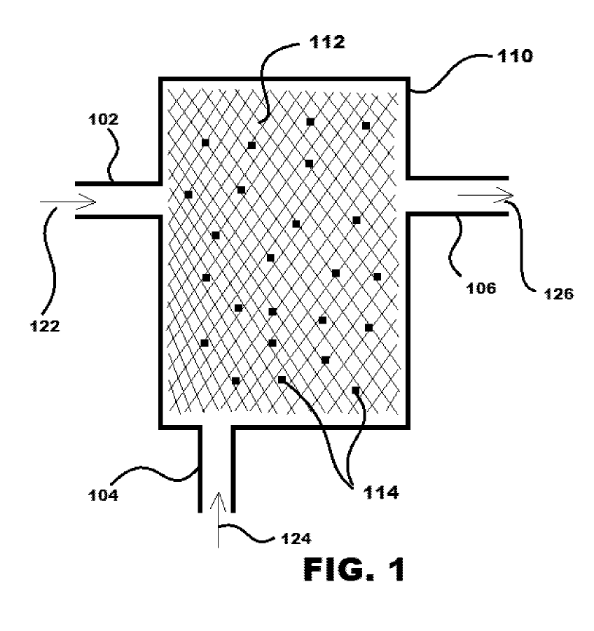

Referring to FIG. 1, in some cases, a single microfluidic chamber 110 can be used for mixing a biological sample 122 with anticoagulant (not shown, but held by fibrous matrix 112), capturing a target on target capture agent particles 114, and delivering a lysis buffer 124 to lyse the target to produce a lysate 126 for analysis. As shown, biological sample 122 can enter microfluidic chamber 110 via inlet port 102. As shown, lysis buffer 124 can enter microfluidic chamber 110 via reagent port 104. In some cases, not shown, both a biological sample 122 and a lysis buffer 124 can be introduced to microfluidic chamber 110 via a common inlet. Lysate 126 can leave microfluidic chamber 110 via outlet 106.

In some cases, a magnetic field can be applied to chamber 110 to control the placement and/or movement of particles 114. In some cases, a magnetic field can be applied to move particles around in chamber 110 to facilitate mixing of a biological sample 122 with anticoagulant within chamber 110. In some cases, particles 114 can be magnetic particles and a magnetic field can be applied to keep particles 114 from exiting chamber 110.

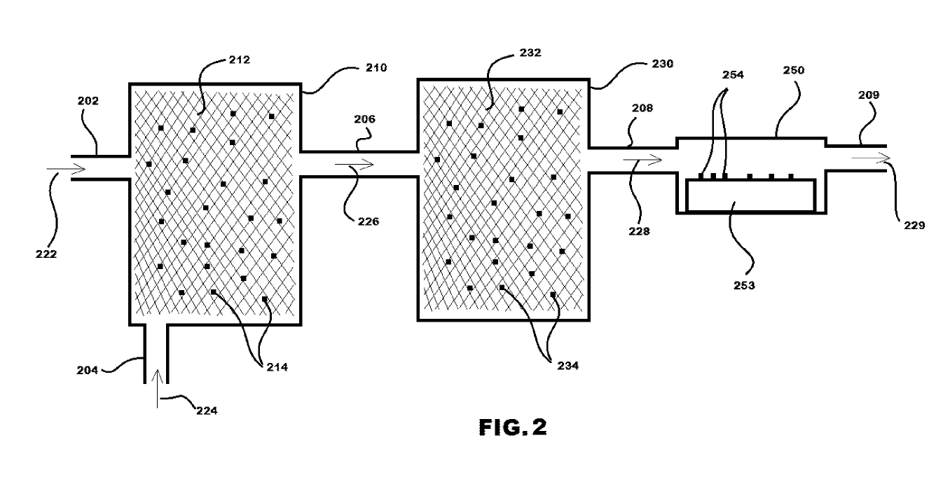

In some cases, multiple microfluidic chambers can be used in series to ensure a more complete capture of target moving through the microfluidic device. For example, FIG. 2 depicts an embodiment including a first microfluidic chamber 210 including a first fibrous matrix 212, a second microfluidic chamber 230 including a second fibrous matrix 232, and a capture chamber 250 including a magnet 253. As shown, a sample 222 (e.g., fingerstick blood sample) is introduced via inlet port 202 to mix with anticoagulant (not shown, but in matrix 212) and target capture agent particles 214. A mixture 226 of sample and anticoagulant then flows through channel 206, through second microfluidic chamber 230, where it can again be mixed with anticoagulant (not shown) and contact target capture agent particles 234. As shown, first and second chambers each include non-woven fibers and particles adapted such that the particles are retained in the fibrous matrix as the sample flows through the microfluidic device. Nonetheless, avoiding the flow of any of particles 214 or 234 can be difficult. Accordingly, in some cases, a final chamber 250 can include magnet 253 to collect particles 254 that become dislodged from first fibrous matrix 210 or second fibrous matrix 230. The collection on magnet 253 of dislodged particles can improve the accuracy of a device used to determine the presence of the target. Although FIG. 2 only includes three chambers 210, 230, and 250, any number of parallel or serial chambers, without or without the fibrous mesh, can be used in methods, devices, and systems provided herein.

Still referring to FIG. 2, lysis buffer 224 can be introduced through a reagent port 204. As the lysis buffer passes through chamber 210, the lysis buffer lyses targets found on particles 214, then the partial lysate 226 passes through channel 206 to second microfluidic chamber 230, where the partial lysate 226 further lyses the captured target bound to particles 234, then lysate 228 passes through channel 208 into collection chamber 250, where dislodged particles 254 can be collected and lysed to form lysate 229. In some case, collection chamber 250 can include a filtering element or other means to prevent particles exiting exit port 209.

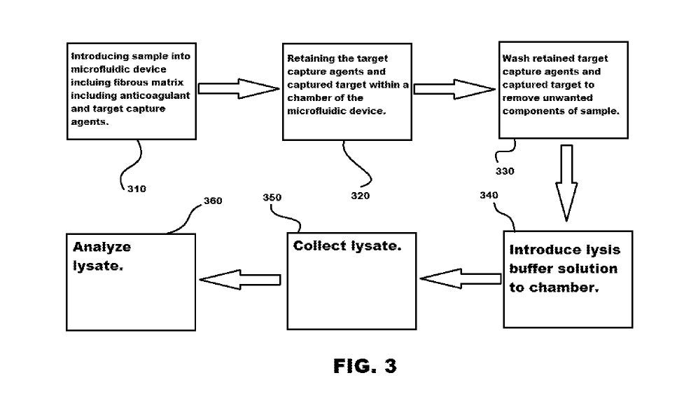

Referring to FIG. 3, a method for determining whether a pathogen or target is present can include introducing 310 a biological sample (e.g., a fingerstick blood test) into an inlet port of a microfluidic device, where the biological sample contacts a fibrous matrix provided herein. As the sample moves through the microfluidic device, a greater number of targets bind to target capture agents 320 to thus keep the target retained in one or more microfluidic or collection chambers. In order to reduce the opportunity for irrelevant blood constituents to interfere with the analysis of the target, a rinse buffer can be delivered 330 through the one or more microfluidic chambers to push the non-captured sample constituents through the microfluidic device. After the rinse, a lysis buffer agent is delivered 340 to the microfluidic chambers and/or the collection chamber to lyse the captured targets and produce a lysate, which can be collected 450, and analyzed 460 to detect the presence or absence of the target. In some cases, analysis of the lysate 460 can indicate an amount of targets found (e.g., a viral load). In some case, a doctor or health care professional may utilize data from devices, systems, and methods provided herein to assist with a diagnosis.

Fibrous Matrix

A fibrous matrix provided herein can be any suitable entanglement of fibers. A fibrous matrix can allow a sample to be wicked through a microfluidic chamber to intermix and/or contact anticoagulant, target capture agents, or a combination thereof. In some cases, methods, devices, and systems provided herein incorporate a non-woven web as the fibrous matrix. Non-woven webs in methods, systems, and devices provided herein can be prepared using any suitable material and any suitable process. Anticoagulant and/or target capture agents may be mixed with structural fibers forming the fibrous matrix during any point in the various processes of processing, producing, and/or further manipulating the structural fibers to produce the anticoagulant/target capture agent entangled fibrous matrix. Suitable methods include the dry laid system, spun bond systems, spun laced systems, melt blown systems, and e-spun systems.

In some case, a fibrous matrix provided herein can be made using a dry laid system. A dry laid system can arrange preformed structural fibers into a web. The preformed structural fibers can be between 1.2 and 100 cm long. For example, the preformed structural fibers can be polyester or polypropylene. In some cases, anticoagulant and/or target capture agents can be mixed in with the structural fibers during fiber processing or just prior to input into a web forming apparatus.

During a dry laid process, preformed fibers (e.g., polypropylene) can be mechanically and/or pneumatically processed from a bale to a point where the fibers can be introduced into a web-forming machine. A dry laid process can include the following steps: bale opening; blending; coarse opening; fine opening; and web-form feeding. During these processes, pins can be used to open fiber tufts in preparation for forming a web. Rolls can also reduce the tuft size by using the principle of carding points between the different rolls The opened fiber with the reduced tufts can be transferred via an air stream to a web-former. In some cases, anticoagulant and/or target capture agents is mixed with the opened fibers just before being fed into a web-former.

One dry laid method of forming a nonwoven web is carding. The carding process separates small tufts into individual fibers, begins to parallelize the fibers, and forms the fibers into a web. In the carding process, fibers are held by one surface while another surface combs the fibers causing individual fiber separation. A large rotating metallic cylinder covered with card clothing can be used to card preformed fibers. The card clothing can include needles, wires, or fine metallic teeth embedded in a heavy cloth or in a metallic foundation. The top of the cylinder may be covered by alternating rollers and stripper rolls in a roller-top card. The fibers, optionally mixed with anticoagulant and/or target capture agents, can be fed by a chute or hopper and condensed into the form of a lap or batting. Needles of the two opposing surfaces of the cylinder and flats or the rollers can be inclined in opposite directions and move at different speeds. The fibers are aligned in the machine direction and form a coherent web below the surface of the needles of the main cylinder. The web can be removed from the surface of cylinder and deposited on a moving belt.

Another dry laid method of forming a nonwoven web is garnetts. Garnetts uses a group of rolls placed in an order that allows a given wire configuration, along with certain speed relationships, to level, transport, comb and interlock fibers to a degree that a web is formed. Garnetts can deliver a more random web than carding.

An air-stream can also be used to orient the structural fibers in a carding or garnetts system. For example, starting with a lap or plied card webs fed by a feed roller, the fibers can be separated by a licker-in or spiked roller and introduced into an air-stream. The air-stream can randomize the fibers as they are collected on the condenser screen. The web can be delivered to a conveyor for transporting to a bonding area. In some cases, the length of fibers used in air-laying varies from 2 to 6 cm. In some cases, the air-stream also delivers a stream of anticoagulant and/or target capture agents to be mixed with the nonwoven fibers.

A centrifugal system can also be used to form a nonwoven web by throwing off fibers from the cylinder onto a doffer with fiber inertia, which is subject to centrifugal force. Orientation in the web is three-dimensional and is random or isotropic. In some cases, anticoagulant and/or target capture agents is added to the centrifugal system to be mixed with the structural fibers.

Web formations can be made into the desired web structure by the layering of the webs from either the card and/or garnetts. Layering techniques include longitudinal layering, cross layering, and perpendicular layering. In some cases, layers of anticoagulant and/or target capture agents are deposited between layers of carded or garneted preformed fibers. As will be discussed below, the non-woven web can be further processed to entangle or interlock the preformed structural fibers of the web with each other and/or with anticoagulant and/or target capture agents. These processed include needling, needle punching, needle felting, stitch bonding, thermal bonding, ultrasonic bonding, radiation bonding, chemical bonding, air-jet entanglement, spun lace, and hydroentanglement.

In a wet laid web process, structural fibers are dispersed in an aqueous medium. Specialized paper machines can be used to separate the water from the fibers to form a uniform sheet of material, which is then bonded and dried. Wet laid Nonwoven Systems can have high production rate (up to 1000 m/min) and the ability to blend a variety of fibers from papermaking technology. Any natural or synthetic fiber could be used in the production of wet-laid nonwovens. For example, cotton linters, wood pulp, and cellulose structural fibers can be used in wet-laid process. Synthetic fibers (e.g., rayon, polyester) can be used and can provide thermobonding capabilities. In some cases, the fibers are between 2 mm and 50 mm long. The wet-laid nonwoven system can use consistencies of between 0.005% and 0.05%. In some cases, anticoagulant and/or target capture agents is also suspended in the water with structural fibers.

After swelling and dispersion of the fibers in water, the mixing vats can be transported to the head box from where they are fed continuously into a web-laying machine. In some cases, anticoagulant and/or target capture agents is also added to the mixing vats prior to feeding the dispersion to the web-laying machine. The anticoagulant and/or target capture agents can be treated prior to adding them to the dispersion. Squeezing machines can be used to dehydrate the web. The web can then be dried and bonded. For example, convection, contact and radiation dryers can be used to both dry and bond the web.

Bonding agents can be added to the wet laid material to help bond the structure. For example, meltable fibers can also be used or added to the web for bonding and activated by a heating step either during drying, or during a later hot calendaring step. Examples of fibers of this type include vinyon, polypropylene, cellulose acetate, and special low melting polyester or polyamide copolymers. In other embodiments, beads of globules of meltable materials can be added during the dry laid process and activated by a heating step to result in spot bonding.

Other in-line treatments can include aperturing and water jet entanglement. Apertures are regularly spaced holes, and can be selected for performance. One method of aperturing uses a course forming wire, so that the sheet is formed around the protruding “knuckles” in a regular pattern. Another method uses high-pressure water showers and patterned cylinders to rearrange the fiber into the desired pattern, which can be used to entangle the fibers and/or create apertures. A process sometimes known as spun lace can use precise jets of high pressure water to hydroentangle the structural fibers with each other and/or with anticoagulant and/or target capture agents. Other processes, including those discussed below, can also be used with a web laid web to entangle anticoagulant and/or target capture agents and/or to bond the structural fibers such that the non-woven web is cohesive.

Polymer-based systems for producing polymeric structural fibers having a nonwoven structure include, for example, melt-blown systems and spun bond systems. Other systems for producing polymeric fibers include electro spinning and force spinning. Moreover, other systems for producing polymeric structural fibers are also contemplated.

Both melt-blowing and spun bonding processes extrude polymeric materials and attenuate (stretch) the extruded polymer to produce fibers. The extruded and attenuated fibers can be collected on a vacuum drum or a conveyor. These processes can be run in both a horizontal and a vertical orientation. Spun bond or melt-blown structural fibers can then be collected on a wind up reel for later entangling with anticoagulant and/or target capture agents. In some cases, anticoagulant and/or target capture agents can be placed in contact with and/or entangled with the spun bond or melt-blown structural fibers during the spun bond or melt-blowing processes. In some cases, target capture agent can be added to the polymeric material before it is melt-blown or spun bond such that the resulting melt-blown or spun bond fibers include the particles at least partially encapsulated by the polymeric material of the structural fibers.

The spun bond and melt-blown processes are somewhat similar from an equipment and operator's point of view and anticoagulant and/or target capture agents can be added to these processes in substantially similar manners. The two major differences between a typical melt-blown process and a typical spun bond process are: i) temperature and volume of the air used to attenuate the filaments; and ii) the location where the filament draw or attenuation force is applied. A melt-blown process uses relatively large amounts of high-temperature air to attenuate the filaments. The air temperature can be equal to or slightly greater than the melt temperature of the polymer. In contrast, the spun bond process generally uses a smaller volume of air close to ambient temperature to first quench the fibers and then to attenuate the fibers. In the melt-blown process, the draw or attenuation force is applied at the die tip while the polymer is still in the molten state. Application of the force at this point can form microfibers but does not allow for polymer orientation. In the spun bond process, this force is applied at some distance from the die or spinneret, after the polymer has been cooled and solidified. Application of the force at this point provides the conditions necessary for polymer orientation, but is not conducive to forming microfibers.

Fibers in fibrous matrix can include the full array of extrudable polymers, such as polypropylene, polyethylene, PVC, viscose, polyester, and PLA. In some cases, the structural fibers have low extractables and/or are biologically inert.

In some cases, anticoagulant and/or target capture agents can be blown by a blower into a stream of melt-blown or spun bond structural fibers exiting a die in a horizontal process. The stream of anticoagulant and/or target capture agents entangled with the structural fiber can be collected and calendared between a pair of vacuum drums. Calendaring can be used in combination with heat (either added or latent) to bond the structural fibers. In some cases, additional methods of bonding or entangling the structural fibers can be used in fibrous matrix.

In some cases, the anticoagulant and/or target capture agents/fibrous matrix can further processed to further secure the anticoagulant and/or target capture agents within the fibrous matrix. For example, the fibrous matrix composite may be needled, needle punched, needle felted, air jet entangled, spun laced, or hydroentangled.

Anticoagulants

As discussed above, methods, systems, and devices can include anticoagulants retained in a fibrous matrix to simplify the mixing of a biological sample with the anticoagulant. Any suitable anticoagulant can be included in methods, systems, and devices provided herein. In some cases, the anticoagulant can be an antithrombics, fibrinolytic, thrombolytics, or a combination thereof. In some cases, an anticoagulant in a method, system, or device provided herein includes ethylenediamine tetraacetic acid (EDTA), heparin, Warfarin (Coumadin), Acenocoumarol, phenprocoumon, Atromentin, Brodifacoum, Phenindione, or a combination there.

Anticoagulants can be incorporated into the fibrous matrix using any suitable method. In some case a solution of anticoagulant is added to a non-woven sheet and dried. In some cases, particles of anticoagulant can be incorporated into the fibrous matrix.

Target Capture Agents

Target capture agents can be incorporated a fibrous matrix to capture a desired target to separate the target form remaining biological constituents. Accordingly, the selection of the target capture agent is highly dependent on the target. For example, in some cases, the target is a virus and the target capture agent is a virion capture agent. Suitable target capture agents include anit apoE ab1, anti apoE ab2, anti apoE ab3, anti apoE ab3, anti E2 ab2, anti E2 ab4, heparin, E2 aptamer, DC-SIGN-Fc chimea, protein G mag beads, streptavidin mag beads, Ni-NTA mag beads, apoH mag beads, MBP-6×His-no CaCl₂, and combinations thereof.

Alternative Arrangements

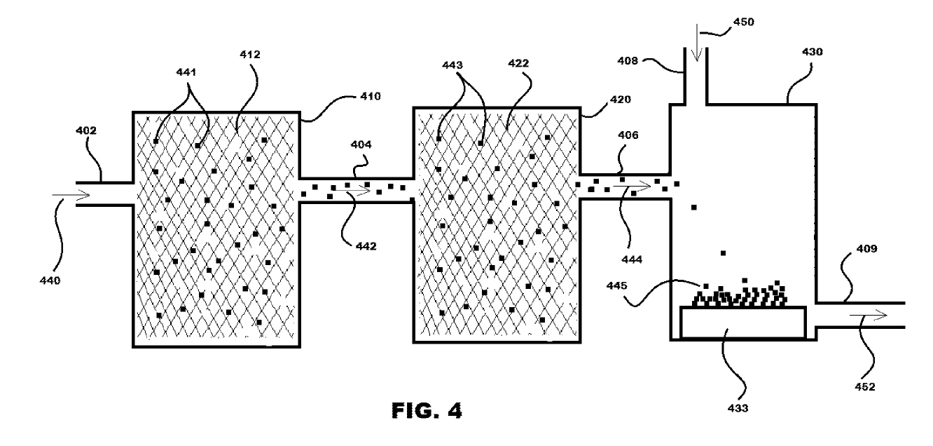

Referring to FIG. 4, methods, devices, and systems provided herein can have particle sizes and pore sizes suitable to allow for particles to flow in and out of the fibrous matrix and between chambers. As shown, three microfluidic chambers are connected. First microfluidic chamber 410 and second microfluidic chamber 420 each include a fibrous matrix 412 and 422 including anticoagulant (not shown) and particles 441 and 443. A biological sample 440 can be received through inlet port 402, mix with anticoagulant retained by fibrous matrix 412, and contact particles 441. A mixture of sample and particles 442 flow in channel 404 to second microfluidic chamber 420, and continues to create a flow of particles through the microfluidic channel 406 to collection chamber 430, which particles 445 are collected with magnet 433. A rinse buffer can be delivered through the apparatus through inlet port 402 and out outlet port 409. Between the movement of the sample and the rinse, a majority of the particles moves into collection chamber 430 during use. A lysis buffer 450 can be delivered through inlet port 408 in collection chamber 430 to lyse the particles in collection chamber 430, and create a lysate 452 which exits outlet port 409. The particles can, in some cases, be metallic and/or magnetic.

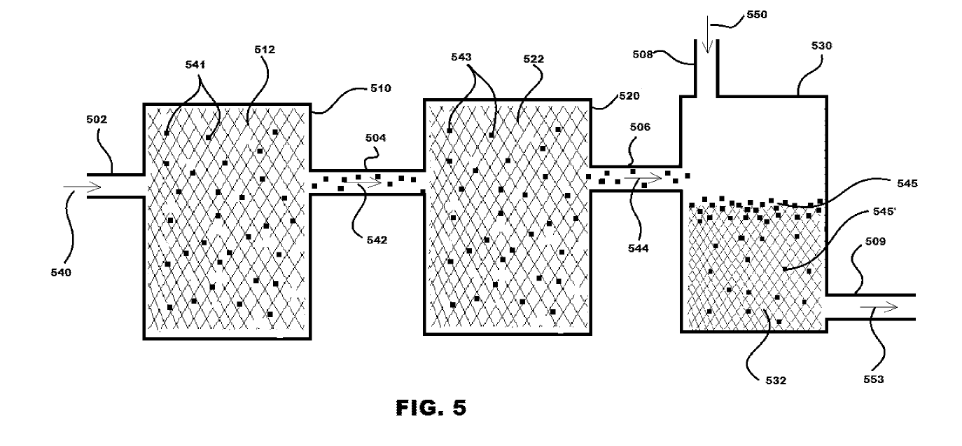

Referring to FIG. 5, methods, devices, and systems provided herein also can have particle sizes and pore sizes suitable to allow for particles to flow in and out of the fibrous matrix and between chambers. As shown, three microfluidic chambers are connected. Each microfluidic chamber 510, 520, and 530 includes a fibrous matrix 512, 522, and 532 including anticoagulant (not shown) and particles 541 and 543. A biological sample 540 can be received through inlet port 502, mix with anticoagulant retained by fibrous matrix 512, and contact particles 541. A mixture of sample and particles 542 flow in channel 504 to second microfluidic chamber 520, and continues to create a flow of particles through the microfluidic channel 506 to collection chamber 530, which particles 545 are collected in fibrous matrix 532. A rinse buffer can be delivered through the apparatus through inlet port 502 and out outlet port 509. Between the movement of the sample and the rinse, a majority of the particles moves into collection chamber 530 during use. A lysis buffer 550 can be delivered through inlet port 508 in collection chamber 530 to lyse the particles in collection chamber 530, and create a lysate 552 which exits outlet port 509. The particles can, in some cases, be metallic and/or magnetic.

In some cases, the devices, systems, and methods provided herein relate to diagnosing one or more disease conditions (e.g., HIV infections, syphilis infections, malaria infections, anemia, gestational diabetes, and/or pre-eclampsia). For example, a biological sample (e.g., blood) can be collected from a mammal (e.g., pregnant woman) and analyzed using a kit including a cartridge including one or more deformable reservoirs provided herein, each reservoir including a reagent, such that the reagent can be mixed with the biological sample using a controller that receives the cartridge to determine whether or not the mammal has any of a group of different disease conditions. In the case of a device that diagnoses multiple disease conditions, the analysis for each disease condition can be performed in parallel, for example using different reagents from different deformable reservoirs, such that the results for each condition are provided at essentially the same time. In some cases, the devices, systems, and methods provided herein can be used outside a clinical laboratory setting. For example, the devices, systems, and methods provided herein can be used in rural settings outside of a hospital or clinic. Any appropriate mammal can be tested using the methods and materials provided herein. For example, dogs, cats, horses, cows, pigs, monkeys, and humans can be tested using a diagnostic device or kit provided herein.

A number of embodiments of the invention have been described. Nevertheless, it will be understood that various modifications may be made without departing from the spirit and scope of the invention. Accordingly, other embodiments are within the scope of the following claims.

What is claimed is:

1. A target detection device for analyzing a biological sample comprising a microfluidic device, the microfluidic device comprising a biological sample inlet in fluid communication with at least a first microfluidic chamber, the at least a first microfluidic chamber containing a fibrous matrix, the fibrous matrix carrying anticoagulant, target capture agents, or a combination thereof within the fibrous matrix.

2. The target detection device of claim 1, wherein the fibrous matrix comprises a non-woven web.

3. The target detection device of claim 1, wherein the fibrous matrix comprises polypropylene fibers.

4. The target detection device of claim 1, wherein the fibrous matrix comprises polyester fibers.

5. The target detection device of claim 1, wherein the target capture agents are bonded to a plurality of particles dispersed within the fibrous matrix.

6. The target detection device of claim 5, wherein said particles are nanoparticles.

7. The target detection device of claim 5, wherein said particles have an average particle diameter of between 100 nm and 1 μm.

8. The target detection device of claim 5, wherein the particles are adapted to have at least 50% of the particles retained in the fibrous matrix as biological samples, rinsing buffers, and lysis buffers are passed through the fibrous matrix to contact the particles.

9. The target detection device of claim 8, where said particles have an average diameter that is larger than an average pore size of said fibrous matrix.

10. The target detection device of claim 5, wherein the fibrous matrix and the particles are needle punched.

11. The target detection device of claim 5, wherein the fibrous matrix is point bonded.

12. The target detection device of claim 5, further comprising at least a second microfluidic chamber in fluid communication with said at least first microfluidic chamber, the at least a second microfluidic chamber comprising a magnet, wherein the particles are adapted to be attracted to the magnet.

13. The target detection device of claim 5, further comprising a magnet adapted to retain the particles within at least one microfluidic chamber.

14. The target detection device of claim 1, further comprising a second microfluidic chamber in fluid communication with said at least first microfluidic chamber, the at least a second microfluidic chamber comprising a second fibrous matrix.

15. The target detection device of claim 14, wherein the second fibrous matrix carries anticoagulant, target capture agents, or a combination thereof.

16. The target detection device of claim 1, wherein the target detection agent is selected from the group consisting of anit apoE ab1, anti apoE ab2, anti apoE ab3, anti apoE ab3, anti E2 ab2, anti E2 ab4, heparin, E2 aptamer, DC-SIGN-Fc chimea, protein G mag beads, streptavidin mag beads, Ni-NTA mag beads, apoH mag beads, MBP-6×His-no CaCl2, and combinations thereof.

17. The target detection device of claim 1, wherein the anticoagulant is selected from the group consisting of ethylenediamine tetraacetic acid (EDTA), heparin, and combinations thereof

18. The target detection device of claim 1, wherein the microfluidic chamber is adapted to be reversible compressed or expanded.

19. A method for detecting the presence of a target in a biological sample comprising:
introducing a biological sample into a microfluidic device to flow through a fibrous matrix in the microfluidic device, the fibrous matrix carrying anticoagulant and target capture agents, the anticoagulant mixing with the biological sample, any targets in the biological sample bonding to the target capture agents, the target capture agents and any bonded targets remaining in one or more microfluidic chambers of the microfluidic device;
delivering a lysis agent through the one or more microfluidic chambers to lyse the target and produce lysate; and
analyzing the lysate to detect a presence of the target in the biological sample.

20. The method of claim 19, further comprising delivering a rinsing buffer after introducing the biological sample and before delivering the lysis agent to rinse the one or more microfluidic chambers of constituents of the biological sample not bonded to the target capture agents.

21. The method of claim 19, wherein the fibrous matrix is held within the one or more microfluidic chambers.

22. The method of claim 19, wherein said target capture agent is retained within the one or more microfluidic chambers by being bonded to a particle, the particle being entangled within the fibrous matrix, attracted to a magnet, or a combination thereof.

23. The method of claim 19, wherein the biological sample is a blood sample, the anticoagulant is selected from the group consisting of ethylenediamine tetraacetic acid (EDTA), heparin, or a combination thereof, the target capture agent being selected from anit apoE ab1, anti apoE ab2, anti apoE ab3, anti apoE ab3, anti E2 ab2, anti E2 ab4, heparin, E2 aptamer, DC-SIGN-Fc chimea, protein G mag beads, streptavidin mag beads, Ni-NTA mag beads, apoH mag beads, MBP-6×His-no CaCl2, and combinations thereof, and the fibrous matrix comprises a non-woven web of polypropylene fiber, polyester fibers, or a combination thereof.

24. The method of claim 19, further comprising changing the volume of the microfluidic chamber.
